(19) United States
(12) Patent Application Publication
(10) Pub. No.: US 2012/0209255 A1
(43) Pub. Date: Aug. 16, 2012

(54) MEDICAL INSTRUMENT

(76) Inventors: Martin Blocher, Stockach-Espasingen (DE); Uwe Bacher, Tuttlingen (DE)

(21) Appl. No.: 13/397,432

(22) Filed: Feb. 15, 2012

(30) Foreign Application Priority Data
Feb. 15, 2011 (DE) 10 2011 011 244.8

(51) Int. Cl. A61B 17/00

(52) U.S. Cl. 606/1

(57) ABSTRACT

A medical instrument with a hollow shaft and a handle mounted on the proximal end of the shaft, such that the shaft can be rotated around its longitudinal axis by a power drive, and such that the power drive is configured as an adjusting wheel that can both be rotated around the longitudinal axis of the shaft and also moved in the longitudinal direction of the shaft. To provide a medical instrument whose power drive makes simple and secure operation of the shaft rotation possible, the power drive can be actuated in every working position in one-handed operation, independently of any other actuation of the handle.

### CROSS-REFERENCE TO RELATED APPLICATIONS

The present application claims priority of German patent application No. 10 2011 011 244.8 filed on Feb. 15, 2011, the content of which is incorporated herein by reference.

### FIELD OF THE INVENTION

The invention relates to a medical instrument with a hollow shaft and a handle mounted on the proximal end of the shaft, such that the shaft can be rotated around its longitudinal axis by a power drive and such that the power drive is configured as an adjusting wheel that can both rotate around the longitudinal axis of the shaft and can also be displaced in the longitudinal direction of the shaft.

### BACKGROUND OF THE INVENTION

With medical instruments that have a curved shaft, it is advantageous for the shaft to be rotatable around the longitudinal axis of the shaft by its power drive in order to ensure, without pivoting the holding hand, a constantly optimal alignment of the medical instrument with respect to the surgical site.

A generic medical instrument is known, for example, from patent DE 102 14 810 A1. This instrument configured as a forceps comprises a shaft 4 on whose proximal end a handle is configured and on whose distal end a forceps jaw is configured. To be able to rotate the forceps jaw around the longitudinal axis, a rotary wheel is positioned on the distal end of the handle. By rotating the rotary wheel, the shaft and the forceps jaw can be rotated around the longitudinal axis. In addition, this forceps comprises a rotary stop between the rotary wheel and the housing of the handle that is configured as a two-sided ring-shaped serration.

This rotary stop comes into effect only when the pivotable gripping member of the handle is actuated to actuate the forceps jaw. This action serves to prevent the forceps jaw in the closed gripping position from being rotated around the longitudinal axis. In practice, however, it is necessary to some extent to modify the position of the jaw members while they are in the closed position, which, with this known instrument, can occur only by rotating the holding hand because of the rotary stop.

An additional medical instrument is known, for example, from patent DE 37 11 377 C2. With this known instrument, the power drive is configured as an adjusting wheel that can rotate together with the shaft around the shaft longitudinal axis. To enable the selection of individual rotary steps, a spring-loaded ball is provided that engages in corresponding recesses in the adjusting wheel. This construction familiar in the art makes simple rotating of the shaft possible even in one-hand operation, but at the same time the spring-loaded catch-ball does not offer sufficient security from accidental rotation of the shaft.

### SUMMARY OF THE INVENTION

It is consequently the object of the invention to provide a medical instrument of the aforementioned type, whose power drive allows a simple and safe operation of the shaft rotation.

This fulfillment of this object is characterized by the totality of the characteristics of claim 1; in particular, the power drive can be actuated in every working position in one-hand operation, independently of any other type of actuation of the handle.

Owing to the inventive configuration of the power drive as an adjusting wheel that can both rotate and slide axially and that can be actuated in every working position in one-hand operation independently of any other type of actuation of the handle, it is possible for the first time, on the one hand, to ensure reliable blocking of the adjusting wheel in the particular rotation position of the shaft and, on the other hand, to be able to rotate the shaft even when the handle has already been actuated.

With a first embodiment of the invention, it is proposed that the power drive should be positioned in the area of the distal end of the handle.

According to an alternative second inventive embodiment, it is proposed that the power drive should be positioned in the area of the proximal end of the shaft. In both cases it is necessary that the power drive, for example by means of a friction-locked or form-fitted connection, should interact with the shaft in such a way that by actuating the power drive, for example rotating the adjusting wheel, the shaft can be rotated around its longitudinal axis.

According to a practical embodiment of the invention, it is proposed that the adjusting wheel should be capable of displacement against the force of at least one spring element in the longitudinal direction of the shaft in order to provide additional security against accidental rotation of the adjusting wheel and thus also of the shaft. The adjusting wheel is preferably pre-tensed in the direction toward the position that blocks the shaft, so that the adjusting wheel automatically returns to the blocking position when force is no longer exerted on the adjusting wheel in the axial direction.

Blocking the adjusting wheel in the particular rotation position of the shaft occurs, according to the invention, by means of catch-lock elements that are configured on the adjusting wheel and on the handle and correspond with one another, by means of which the shaft can be fixed on the handle to rotate in predetermined catch-lock steps around the longitudinal axis of the shaft.

With a preferred embodiment of the invention, it is proposed that the catch-lock elements should be positioned coaxially around the longitudinal axis of the shaft, such that the catch-lock elements are configured as catch-pins or catch-noses positioned on the adjusting wheel and as catch-grooves configured on the handle.

According to a practical embodiment of the invention, the catch-grooves are configured in a catch-disc positioned on the distal end of the handle.

Finally, it is proposed with the invention that the shaft should be configured, at least in some portions, as curved at an angle with respect to the longitudinal axis of the shaft.

To position the power drive that is required for the rotation of the shaft, the invention foresees two embodiments, namely the arrangement of the power drive in the area of the distal end of the handle or alternatively the arrangement of the power drive in the area of the proximal end of the shaft.

Further characteristics and advantages of the invention can be seen from the appended drawings, in which two embodiments of an inventive medical instrument are illustrated only by way of example, without restricting the invention to these embodiments.

### BRIEF DESCRIPTION OF THE DRAWINGS

FIG. 1 shows a side view of an inventive medical instrument.

FIG. 2 shows a section along the line II-II from FIG. 1.

FIG. 3 shows an enlarged depiction of detail III from FIG. 2, showing the power drive in the blocked position.

FIG. 4 shows a depiction according to FIG. 3, but showing the power drive in the rotatable position.

FIG. 5 shows an enlarged depiction of a first embodiment of an inventive adjusting wheel.

FIG. 6 shows an enlarged depiction of a second embodiment of an inventive adjusting wheel.

### DETAILED DESCRIPTION OF THE INVENTION

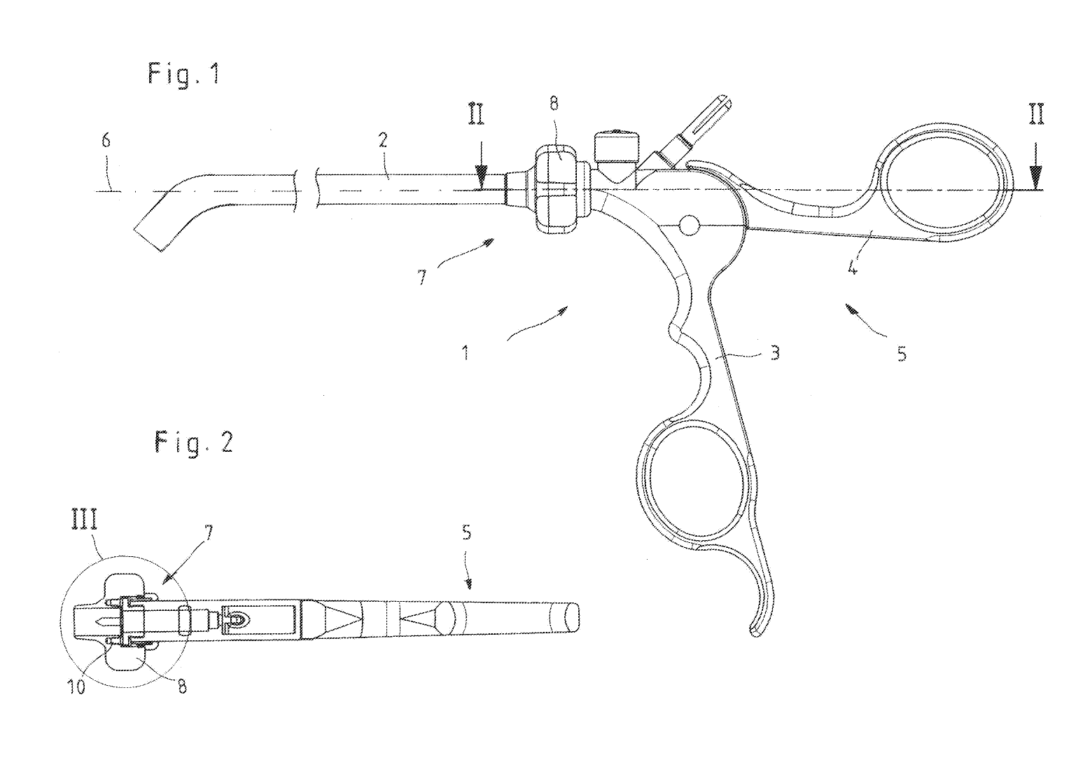

The medical instrument 1 for endoscopic purposes shown in FIG. 1 consists essentially of a hollow shaft 2 on whose proximal end a handle 5 is mounted that consists of two gripping members 3 and 4. As can also be seen from FIG. 1, the shaft 2 is configured in the area of its distal end as curved at an angle with respect to the longitudinal axis 6 of the shaft 2.

In order to ensure a constantly optimal alignment of the curved shaft 2 of the medical instrument 1 with respect to the surgical site without rotating the holding hand, the shaft 2 is mounted on the handle 5 so that it can rotate around its longitudinal axis 6 by means of a power drive 7. Thus the power drive 7 is positioned in the area of the distal end of the handle 5 in such a way that the user of the medical instrument 1 can operate the power drive 7 in one-hand operation with the holding hand gripping the handle 5.

It can also be useful to rotate the shaft 2 of the medical instrument 1 around its longitudinal axis 6 in the case of a straight shaft 2, that is, a shaft 2 without distal curvature, when for example the distal end of the shaft 2 is not configured as straight but rather, for example, as bent or spoon-shaped at an angle of less than 45 degrees to the longitudinal axis 6. Also in case of such a non-straight configuration of the distal end of the otherwise straight shaft 2, it can be necessary in practice to align the shaft 2 or its distal end with respect to the handle 5.

In the illustrated embodiment, the power drive 7 is configured as an adjusting wheel 8 mounted on the distal end of the handle 5. Alternatively to this embodiment, it is also possible of course to position the power drive 7 on the proximal end of the shaft 2 in the transition to the handle 5. In all cases it is necessary that the power drive 7, for example by means of a friction-locked or form-locked connection, should interact with the shaft 2 in such a way that, by actuating the power drive 7, for example by rotating the adjusting wheel 8, the shaft can be rotated around its longitudinal axis 6.

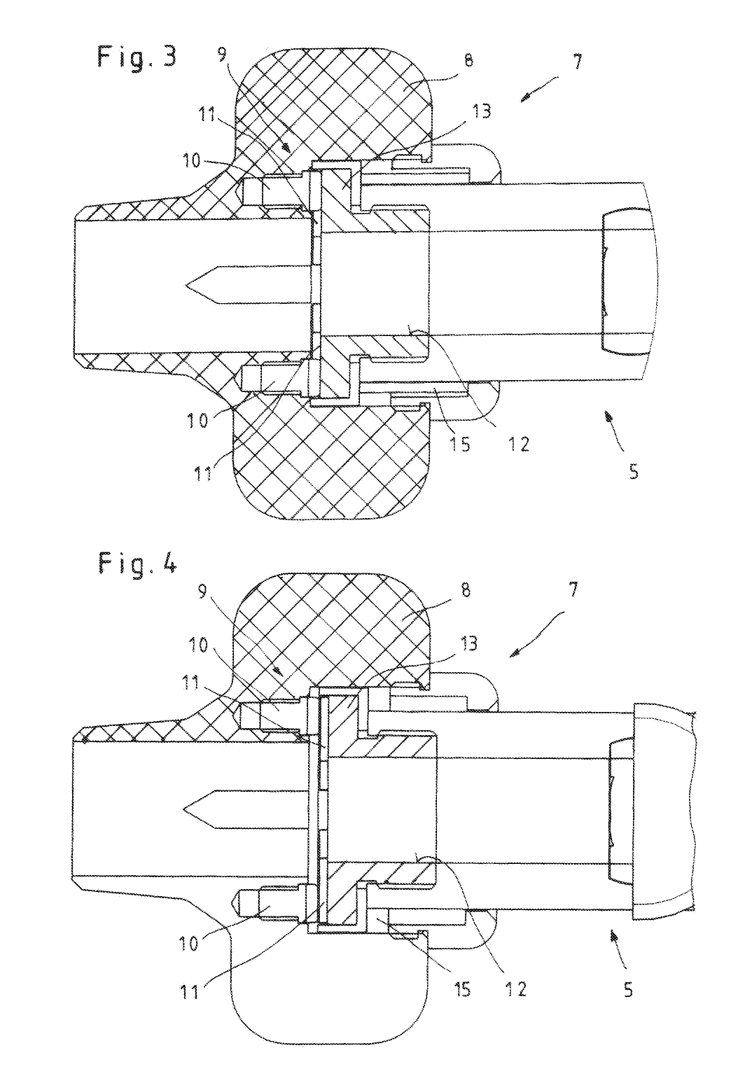

The structure of the power drive 7 can be seen in greater detail in FIGS. 3 and 4, in particular.

The power drive 7 of this medical instrument 1 is distinguished by a particular protective mechanism to prevent unintentional rotation of the adjusting wheel 8 and thus of the shaft 2. This protective mechanism consists in the fact that the adjusting wheel 8 can be moved between a position that releases the rotation of the shaft 2 around the longitudinal axis 6 of the shaft 2 and a position that blocks the shaft 2 in the longitudinal direction of the shaft 2. For this purpose, the power drive 7 is configured both so that it can rotate around the longitudinal axis 6 of the shaft 2 and also so that it can move in the longitudinal direction of the shaft 2.

Only in the position of the adjusting wheel 8 displaced in the axial direction of the shaft 2 is the adjusting wheel 8 rotatable around the longitudinal axis 6 of the shaft 2 along with the shaft 2.

Blocking the shaft 2 on the handle occurs by means of catch-lock elements 9 that are configured on the adjusting wheel 8 and on the handle 5 and correspond with one another, and by which the shaft 2 can be fixed on the handle 5 to rotate in predetermined catch-lock steps around the longitudinal axis 6 of the shaft 2.

In the embodiment shown in FIGS. 3 and 4, the catch-lock elements 9 are configured as catch-pins 10 mounted on the adjusting wheel 8 and catch-grooves 11 configured on the handle 5, such that the catch-lock elements 9 are positioned coaxially around the longitudinal axis 6 of the shaft 2, because the hollow shaft 2 continues in a pass-through borehole 12 in the handle 5, in order to be able to insert other medical instruments into the hollow shaft 2 from the proximal end of the medical instrument 1.

The catch-grooves 11 for receiving the catch-pins 10 are configured in the illustrated embodiment in a catch-disc 13 attached on the distal end of the handle 5.

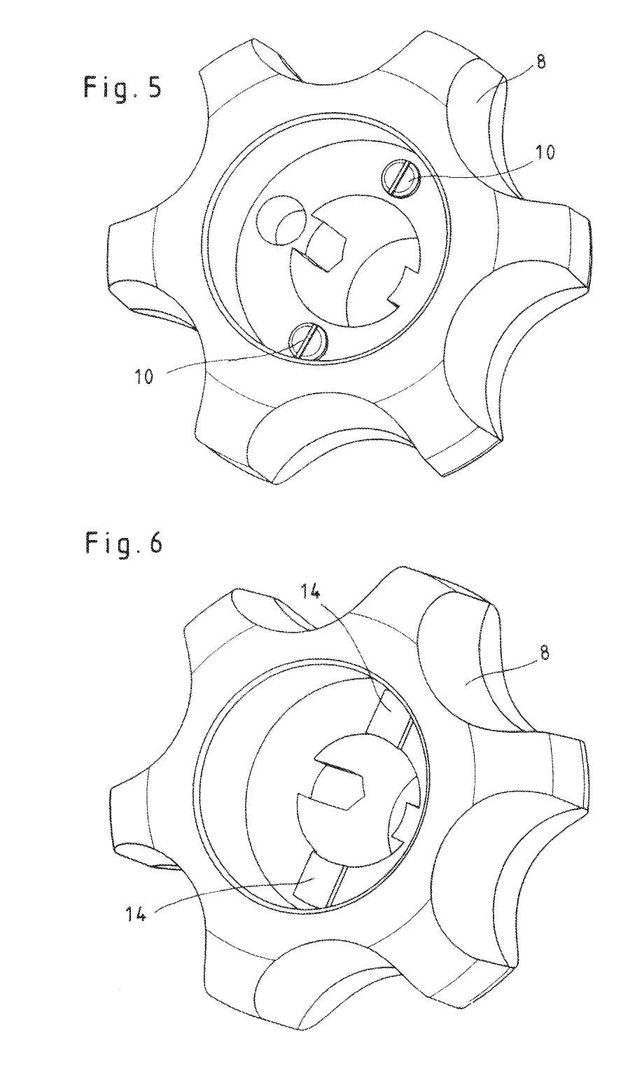

FIGS. 5 and 6 show two embodiments for configuring the catch-lock elements 9 that correspond with the catch grooves 11. The first embodiment, shown in FIG. 5, corresponds to the depiction according to FIGS. 3 and 4, in which the catch-lock elements 9 on the adjusting wheel side are configured as catch-pins 10. In the second embodiment, illustrated in FIG. 6, these catch-lock elements 9 are configured as catch-noses 14 that correspond with the catch-grooves 11.

As an alternative to the illustrated embodiments, it is also possible of course to reverse the arrangement of the catch-lock elements 9, that is, to configure the catch-pins 10 or catch-noses 14 on the handle 5 and the catch-grooves 11 along with the catch-disc 13 on the adjusting wheel 8.

To provide additional security against unintended rotation of the adjusting wheel 8 and thus also of the shaft 2, the adjusting wheel 8 can be displaced against the force of a spring element 15 in the longitudinal direction of the shaft 2. The adjusting wheel 8 is thus pre-tensed by the spring element 15 in the direction toward the position that blocks the shaft 2, so that the adjusting wheel 8 automatically returns to the blocking position when no further force is exerted on the adjusting wheel 8 in the axial direction.

In the configuration just described, the power drive 7 can be actuated in every working position in one-hand operation independently of other actuation of the handle 5, because no reciprocal action exists between the power drive 7 and the actuation of the gripping members 3 and 4 of the handle 5.

Operation of the medical instrument 1 described above, in particular with reference to FIGS. 1, 3 and 4, occurs as follows:

FIG. 3 shows the power drive 7 of the medical instrument 1 in the position that blocks the shaft 2 on the handle 5, the position in which the catch-lock elements 9 of the adjusting wheel 8 that are configured as catch-pins 10 engage in the catch-lock elements 9 that are configured as catch-grooves 11 and positioned in the catch-disc 13.

To move the shaft 2 into a new working position, the user of the medical instrument 1 pushes the adjusting wheel 8 against the force of the spring element 15 in the axial direction toward the distal end until the catch-pins 10 disengage from the catch-grooves 11, as shown in FIG. 4. It is exclusively when the adjusting wheel is in this axially displaced rotary position that the adjusting wheel 8 can now be rotated around the longitudinal axis 6 of the shaft 2 together with the shaft 2 until the shaft 2 has assumed the desired position.

As soon as the user is no longer exerting any pressure in the axial direction on the adjusting wheel 8, the spring element 15 draws the adjusting wheel 8 back toward the proximal end in axial direction until the catch-pins 10 re-engage with the catch-grooves 11 and thus block the shaft 2 again in relation to the handle 5, as shown in FIG. 3.

As an alternative to the illustrated and previously described embodiment, it is also possible of course to reverse the axial displacement of the adjusting wheel 8; that is, the user draws the adjusting wheel 8 in the axial direction toward the proximal end against the force of the spring element 15 to release the adjusting wheel from the blocking position. As soon as the user is no longer pulling in the axial direction on the adjusting wheel 8, the spring element 15 presses the adjusting wheel 8 back toward the distal end in the axial direction until the catch-pins 10 re-engage with the catch-grooves 11 and thus block the shaft 2 again in relation to the handle 5.

Because actuation of the power drive 7, as well as release of the rotary blocking owing to the catch-lock elements 9 and the spring element 15, can occur in every working position in one-hand operation, independently of any other actuation of the handle 5, this medical instrument 1 lends itself to an especially wide range of uses.

A medical instrument 1 configured as described above is characterized in that, in addition to its simple structure and ease of operation, it offers the greatest possible security against unintended rotation of the shaft 2.

1. A medical instrument with a hollow shaft and a handle positioned on the proximal end of the shaft, such that the shaft can be rotated around its longitudinal axis by a power drive and such that the power drive is configured as an adjusting wheel that can both be rotated around the longitudinal axis of the shaft and also be displaced in the longitudinal direction of the shaft,
wherein
the power drive can be actuated in every working position in one-hand operation independently of any other actuation of the handle.

2. The medical instrument according to claim 1, wherein the adjusting wheel can be displaced in the longitudinal direction of the shaft between a position that releases the rotation of the shaft around the longitudinal axis of the shaft and a position that blocks the shaft.

3. The medical instrument according to claim 1, wherein the adjusting wheel can be displaced in the longitudinal direction of the shaft against the force of at least one spring element.

4. The medical instrument according to claim 3, wherein the adjusting wheel is pre-tensed by the at least one spring element in the direction toward the position that blocks the shaft.

5. The medical instrument according to claim 1, wherein catch-lock elements are configured on the adjusting wheel and on the handle and serve to fix the shaft on the handle to rotate in predetermined catch-lock steps around the longitudinal axis of the shaft.

6. The medical instrument according to claim 5, wherein the catch-lock elements are positioned coaxially around the longitudinal axis of the shaft.

7. The medical instrument according to claim 5, wherein the catch-lock elements are configured as catch-pins or catch-noses positioned on the adjusting wheel and as catch-grooves configured on the handle.

8. The medical instrument according to claim 7, wherein the catch-grooves are configured in a catch-disc positioned on the distal end of the handle.

9. The medical instrument according to claim 1, wherein the shaft, at least in portions, is configured as curved at an angle with respect to the longitudinal axis of the shaft.

10. The medical instrument according to claim 1, wherein the power drive is positioned in the area of the distal end of the handle.

11. The medical instrument according to claim 1, wherein the power drive is positioned in the area of the proximal end of the shaft.

12. The medical instrument according to claim 6, wherein the catch-lock elements are configured as catch-pins or catch-noses positioned on the adjusting wheel and as catch-grooves configured on the handle.
